(19) United States
(12) Patent Application Publication
(10) Pub. No.: US 2006/0228251 A1
(43) Pub. Date: Oct. 12, 2006

(54) Pulsed high-intensity light sterilization

(75) Inventors: Gary E. Schneberger, Elkridge, MD (US); Timir Patel, Clarksville, MD (US); Donald J. Rook, Bel Air, MD (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Jagaji Holdings, LLC

(21) Appl. No.: 11/099,498

(22) Filed: Apr. 6, 2005

(51) Int. Cl. A61L 2/08; A61L 2/10

(52) U.S. Cl. 422/23; 422/24; 250/455.11

(57) ABSTRACT

A method and apparatus for terminal sterilization. The method orients a wall of a container in relation to at least one flashlamp, where the wall has an inner surface and an outer surface. The method creates a vortex in a fluid held by the container. The method generates from each flashlamp at least one pulse of high-intensity light in a broad spectrum and exposes the container to each pulse of high-intensity light.

### FIELD OF THE INVENTION

The present invention relates, in general, to irradiation of objects using pulsed high-intensity light. In particular, the present invention is an apparatus and process for irradiating an object with pulsed high-intensity light to sterilize either the object or the object and its contents.

### BACKGROUND OF THE INVENTION

The prior art teaches that irradiation of bacterial, fungal, or mold spores with ultraviolet light in the approximate wavelengths of 254±20 nanometers (the “spectral region of interest (“ROI”)) will kill such flora. The specific mechanisms of kill include disruption of the cell wall, and disintegration of the spores' DNA through scission, fragmentation, and segmentation of the double helix of the DNA. Such changes result in terminal sterilization of an individual spore, that is, the individual spore is non-viable, and incapable of reproduction.

The use of low-intensity ultraviolet light is also known in the prior art as a means of disinfection for water treatment and medical instrument sterilization. Recent technology advancements for water treatment have shown that the introduction of a “Taylor vortex” will increase the efficiency of the disinfection process. A Taylor vortex is created in a viscous fluid in the gap between two concentric, rotating cylinders. In the simplest case, creation of a Taylor vortex involves holding the outer cylinder at rest while rotating the inner container. Hence, water spinning in a Taylor vortex requires less exposure of the water to the low-intensity ultraviolet light to attain the same kill level as water that is not spinning in a Taylor vortex.

Pulsed high-intensity light is known in the prior art to be capable of providing a high level of disinfection, sanitization, and sterilization of devices and surfaces. The most commonly used light for such purposes is broad spectrum light, produced by flashing a lamp of very high-energy intensity. Xenon lamps are capable of delivering such intense energy over a broad spectrum, ranging from extremely low ultraviolet wavelengths to extremely high infrared wavelengths.

The prior art teaches the use of pulsed high-intensity light for the sterilization of the inside surface and outside surface of the seal area of blow/fill/seal vials. The respiratory care medical practice area commonly uses these vials for the delivery of saline as a drug diluent in nebulizers, and for the flushing of mucous from indwelling nasal catheters. This sterilization method is effective in the respiratory care medical practice area because these vials are typically made of Low Density Polyethylene (LDPE) and pulsed high-intensity light has relatively good transmission through thin cross sections of LDPE. This sterilization method is also not as effective in killing microbial matter in the center of the vial because the light energy is diffracted by the vial wall and the fluid. The prior art also teaches the use of pulsed high-intensity light for the sterilization of a product in a container such as a pharmaceutical in a vial.

However, pulsed high-intensity light is not useful for the sterilization of products that have a tendency to absorb and diffract the light in both the spectral ROI as well as other wavelengths. Thus, pulsed high-intensity light is not useful for the sterilization of products that are opaque to wavelengths in the spectral ROI, whether the visual appearance of the product is opaque or clear, or products that exhibit relatively good transmission in the ROI, but have multiple walls, thick cross sections, or convoluted shapes that may diffract the pulsed high intensity light rays. For example, containers manufactured from clarified polypropylene, which appear to be perfectly clear to the human eye, may have a very low transmission coefficient in the spectral ROI. In one specific case, the containers are medical syringes, one made of polycarbonate, the other made of clarified polypropylene. The polycarbonate syringe is perfectly clear with virtually no haze when compared to the clarified polypropylene syringe. However, the polycarbonate syringe has transmission properties in the spectral ROI that render it unsuitable for terminal sterilization and the clarified polypropylene syringe has a much higher transmission rate of wavelengths in the spectral ROI. Furthermore, the transmission rate may differ among grades of clarified polypropylene, and from manufacturer to manufacturer.

Thus, there is a need for a method and apparatus for terminal sterilization using pulsed high-intensity light that increases the efficiency of the sterilization process to allow for a reduction in the transmission coefficient of the pulsed high-intensity light in the spectral ROI. The present invention addresses this need.

### SUMMARY OF THE INVENTION

A method and apparatus for terminal sterilization. The method orients a wall of a container in relation to at least one flashlamp, where the wall has an inner surface and an outer surface. The method creates a vortex in a fluid held by the container. The method generates from each flashlamp at least one pulse of high-intensity light in a broad spectrum and exposes the container to each pulse of high-intensity light.

### BRIEF DESCRIPTION OF THE DRAWINGS

The accompanying figures best illustrate the details of the method and apparatus for terminal sterilization using pulsed high-intensity light, both as to its structure and operation. Reference numbers and designations that are alike in the accompanying figures refer to like elements.

FIG. 1 is a block diagram that illustrates the components for one exemplary embodiment of a terminal sterilization system that uses pulsed high-intensity light.

FIG. 2A and FIG. 2B are block diagrams that illustrates the components for a prototype of the terminal sterilization system 100 shown in FIG. 1.

FIG. 3 is a flow diagram that describes a terminal sterilization process used in the prototype system 200 shown in FIG. 2A and FIG. 2B.

FIG. 4 and FIG. 5 are block diagrams that illustrate the components for production line exemplary embodiments of the terminal sterilization system 100 shown in FIG. 1.

### DETAILED DESCRIPTION OF THE INVENTION

Various means exist for generating a vortex, or vortices, in a fluid. Exemplary means for generating a vortex in a fluid include mechanically rotating a container that holds a fluid, inserting directional baffles within a fluid that is flowing, mechanically stirring a fluid using a stirring mechanism within a container that holds a fluid, or mechanically rotating one or more cylinders of a container having one or more cylinders within a cylinder where the fluid is in the interstices of the cylinders.

The exposure of a fluid to pulsed high-intensity light will terminally sterilize the fluid. But, the total energy necessary to terminally sterilize the fluid will decrease by creating a vortex in the fluid before the exposure to the pulsed high-intensity light. The vortex has a centrifugal force that pushes away from the center of the vortex and into the rapidly spinning fluid that surrounds the vortex. Thus, the centrifugal force pushes any microbial content contained within the fluid toward the inner surface of the wall of the container. This is advantageous for several reasons. First, the centrifugal force decreases the distance between the microorganisms and the pulsed high-intensity light. Since diffraction causes the light energy to decrease as it penetrates further into the fluid, forcing the microorganisms closer to the inner surface of the wall of the container forces those microorganisms to absorb more of the light energy. Second, the centrifugal force may allow for multiple exposures of the microorganism to the pulsed light. Depending on the speed of rotation of the container and the duration of the pulsed light exposure, the microorganisms may rotate before the pulsed light source multiple times during a single exposure to the pulsed light. Third, the centrifugal forces will minimize shadowing. Since the exposure of a microorganism to a light pulse creates a shadow area behind the microorganism, another microorganism in the shadow will not receive any exposure to the light pulse. Thus, by pushing any microorganisms in the fluid to the inner surface of the wall of the container, the centrifugal force minimizes shadowing.

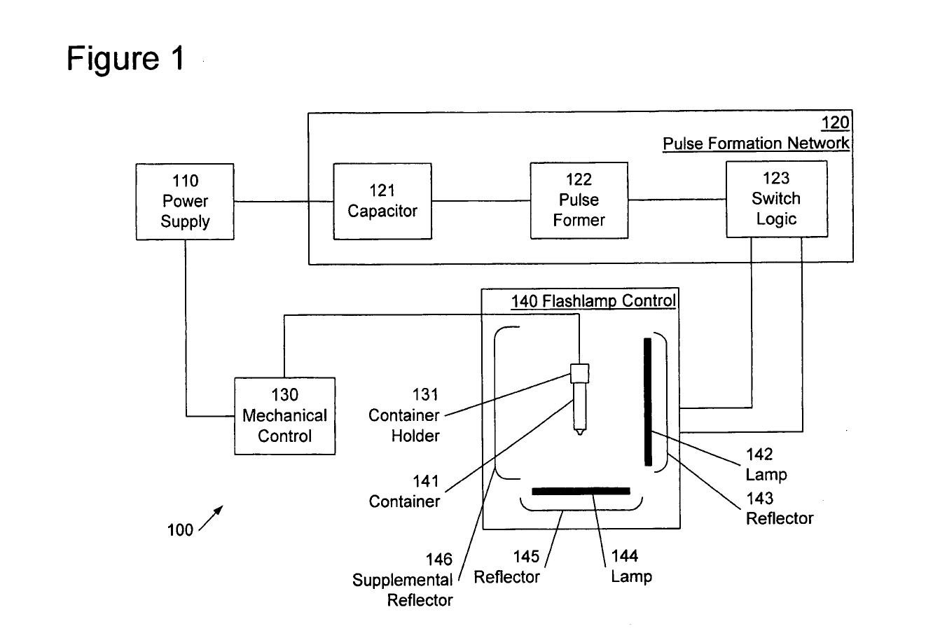

FIG. 1 illustrates the components for one exemplary embodiment of a terminal sterilization system that uses pulsed high-intensity light. The terminal sterilization system 100 comprises a power supply 110, pulse formation network 120, mechanical control 130, and flashlamp control 140. The pulse formation network 120 further comprises a capacitor 121, pulse former 122, and switch logic 123. The flashlamp control 140 further comprises a container 141, container holder 131, lamps 142, 144, reflectors 143, 145, and supplemental reflector 146. The pulse formation network 120 is an exemplary means for generating a pulse of high-intensity light in a broad spectrum.

The power supply 110 generates high voltage electricity to power the pulse formation network 120, mechanical control 130, and flashlamp control 140. The mechanical control 130 is operative to orient the container 141 in relation to the lamps 142, 144, and rotate the container holder 131 and container 141, thus creating a vortex in the fluid held inside the container 141. The mechanical control 130 is an exemplary means for orienting and rotating the container 141. The container 141 may include any object capable of holding the fluid, such as a syringe, vial, test tube, bottle, boxes, bags, or the like. The pulse former 122 generates an electrical pulse when the power supply 110 fully charges the capacitor 121. The switch logic 123 controls the pulsewidth duration of the pulse to the lamps 142, 144 to generate a single flash of high-intensity light. The reflectors 143, 145 and supplemental reflector 146 are operative to reflect divergent rays back toward the focal point of the lamps 142, 144 and/or container 141. The pulse formation network 120 and mechanical control 130 may operate in either a single-fire mode or a continuous mode to saturate the container 141 with pulsed high-intensity light from the lamps 142, 144.

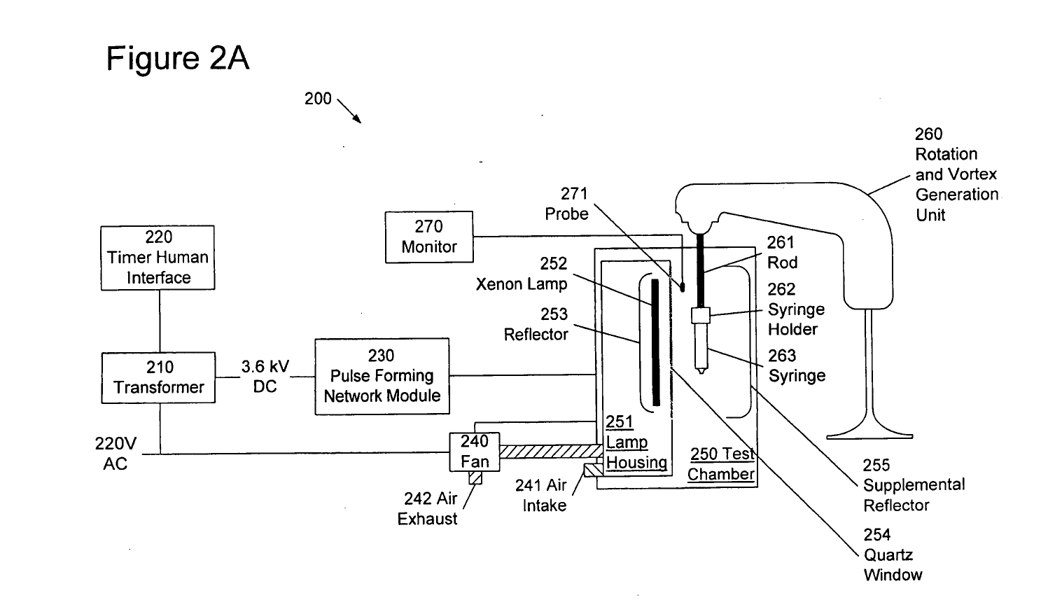

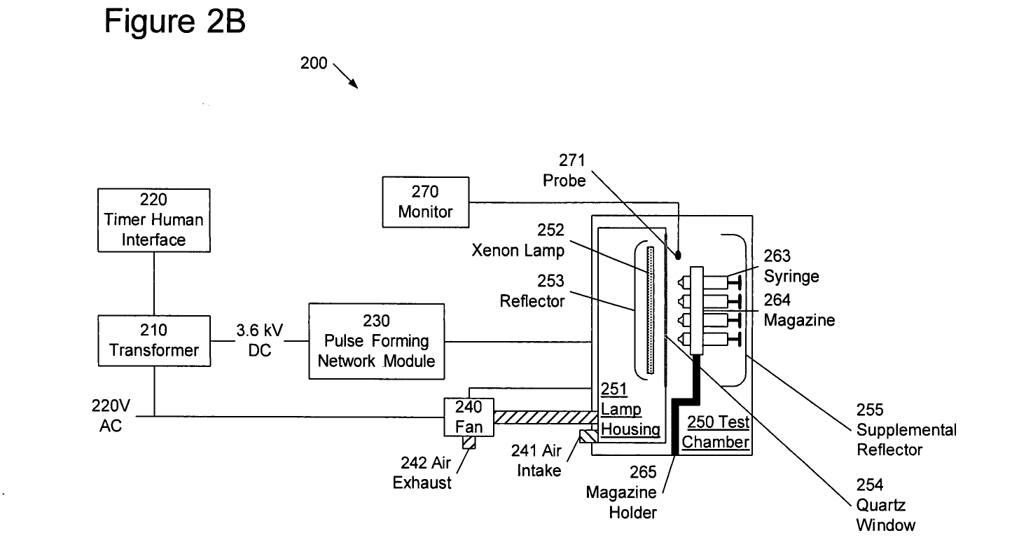

FIG. 2A illustrates the components for a prototype of the terminal sterilization system 100 shown in FIG. 1. The prototype system 200 is suitable for conducting sterilization studies on relatively limited numbers of syringes and is configurable to accommodate other containers or devices. As shown in FIG. 2A, the prototype system 200 uses a 12 cc syringe 263 filled with 10 cc of sterile saline as the container 141. Before processing, a tester inoculates the syringe 263 with a challenge organism, such as Bacillus pumilis at a level of 1×10⁶ to 1×10¹⁰ spores per device. Other suitable challenge organisms exist, including Aspergillus niger, but were not used in the prototype system 200. The filling and inoculation of the syringe 263 follows aseptic techniques. The prototype system 200 comprises a high voltage power supply 210, remote timer human interface 220, pulse forming network module 230, a ventilation and lamp cooling system, custom-built test chamber 250, and light energy monitor 270.

The high voltage power supply 210 further comprises a transformer that receives a 220 V electrical input.

The ventilation and lamp cooling system further comprises a fan 240, air intake 241, and air exhaust 242.

The custom-built test chamber 250 further comprises a lamp housing 251 and supplemental reflector 255. The lamp housing 251 further including a 16-inch pulsed light source (xenon) lamp 252, reflector 253, and 18-inch by 4-inch fused quartz window 254 where the lamp 252 is oriented between a quartz window 254 and reflector 253. The lamp housing 251 also includes connections for electrical supply and ventilation/cooling of the lamp. The test chamber 250 provides an entryway to receive the rod 261, syringe holder 262, and syringe 263 of the rotation and vortex generation unit 260. The remaining items are custom-built components.

The custom-built syringe rotation and vortex generation unit 260 is a mechanical control that rotates the rod 261, syringe holder 262, and syringe 263 to create a vortex in the contents of the syringe 263.

The light energy monitor 270 incorporates a thermopile detector head that includes a probe 271.

In the prototype system 200 shown in FIG. 2A, the manufacturer specified energy output of the pulsed light source (xenon) lamp 252 is 1.27 Joules/cm² at the focal point of the convergent rays, which is located 0.9863 inches from the quartz window 254 of the lamp housing 251. The duration of each pulse of the pulsed light source (xenon) lamp 252 is 200 milliseconds. When running in a continuous mode, the pulsed light source (xenon) lamp 252 is capable of generating three pulses per second. The supplemental reflector 255 is a custom-built component manufactured using optically reflective polished and coated aluminum sheet metal. The arrangement of the syringe 263, pulsed light source (xenon) lamp 252, and supplemental reflector 255 places the syringe 263 between the supplemental reflector 255 and the pulsed light source (xenon) lamp 252. In one exemplary embodiment, the supplemental reflector 255 measures approximately 20 inches in length, 12 inches of internal diameter, with a 5-inch by 19-inch port cut into the bottom of the supplemental reflector 255. The manufacturing of the supplemental reflector 255 also flattens the bottom ½-inch strip of each end of the supplemental reflector 255 to allow the supplemental reflector 255 to fit against the surface of the lamp housing 251 and completely encompass the quartz window 254 with approximately ½-inch clearance. The support for the shape of the supplemental reflector 255 is on the external shell and includes 12-inch round galvanized sheet metal and supportive rings. When using the supplemental reflector 255, the cumulative energy output of the pulsed light source (xenon) lamp 252 and the supplemental reflector 255 increases the energy input to the syringe 263 to approximately 1.6 Joules/cm² at the focal point of the convergent rays, as measured using a monitor 270 and probe 271. The increase in energy input to the syringe 263 from 1.27 Joules/cm² to 1.6 Joules/cm² is most likely attributable to redirection of the reflected divergent rays back to the focal point.

FIG. 2A also illustrates the prototype system 200 configured with a custom-built rod 261 and syringe holder 262 to hold syringe 263. In one exemplary embodiment, the syringe holder 262 comprises a cylindrical holder having a hollow tube to receive the syringe 263. The bottom of the hollow tube includes finger grips for holding the syringe 263. A cap that has an interference fit with the hollow tube holds the syringe 263 in place. This arrangement entirely exposes, axially, the syringe 263 and its contents to the pulsed light source (xenon) lamp 252. The upper end of the syringe holder 262 comprises a hexagonal rod 261 that is approximately 8 inches in length. The hexagonal rod 261 is a means for mounting the syringe 263 and syringe holder 262 to the rotation and vortex generation unit 260.

The rotation and vortex generation unit 260 shown in FIG. 2A comprises a variable speed motor-driven headstock with chuck to accept and lock the rod 261, syringe holder 262, and syringe 263 at the desired height. The rotation and vortex generation unit 260 includes a moveable base to hold the syringe holder 262 and syringe 263 in precisely the desired position within the test chamber 250.

As shown in FIG. 2A, the ventilation and lamp cooling system includes a fan 240, air intake 241, and air exhaust 242. The fan 240 is an impeller-type blower that connects to the lamp housing 251 through the test chamber 250. The air intake 241 uses a 4-inch flex duct to connect to the lamp housing 251 and pull fresh air into the lamp housing 251. The fan 240 connects to the lamp housing 251 to remove the ozone generated when flashing the pulsed light source (xenon) lamp 252 from the test chamber 250. The air exhaust 242 uses a 4-inch flex duct to connect to the lamp housing 251 and ventilate the ozone from the test chamber 250 during testing. In another exemplary embodiment, the ventilation and lamp cooling system may alternatively be connected so that the fan 240 blows coolant air into the lamp housing 251. This alternative arrangement may provide more efficient cooling of the pulsed light source (xenon) lamp 252.

FIG. 2B illustrates another exemplary embodiment of the prototype system 200 shown in FIG. 2A. Since the components comprising the prototype system 200 shown in FIG. 2B are substantially similar to those shown in FIG. 2A, the written description for FIG. 2B will only describe those components that differ from FIG. 2A.

The configuration of the prototype system 200 shown in FIG. 2B includes a custom-built magazine holder 265 and syringe magazine 264 to hold up to four syringes with the cap of each syringe 263 facing the pulsed light source (xenon) lamp 252. The syringe magazine 264 provides direct exposure of the luer tip, or opening, for each syringe 263 to the pulsed high-intensity light from the pulsed light source (xenon) lamp 252, through the cap, with the cap located at the focal point of the convergent rays from the pulsed light source (xenon) lamp 252.

The magazine holder 265, as shown in FIG. 2B, has a first end that attaches to the test chamber 250 and a second end to accept the syringe magazine 264 holding up to four syringes with the cap of each syringe 263 facing the pulsed light source (xenon) lamp 252 at the desired height. The magazine holder 265 positions the syringe magazine 264 and the syringes within the supplemental reflector 146 and in precisely the desired position within the test chamber 250.

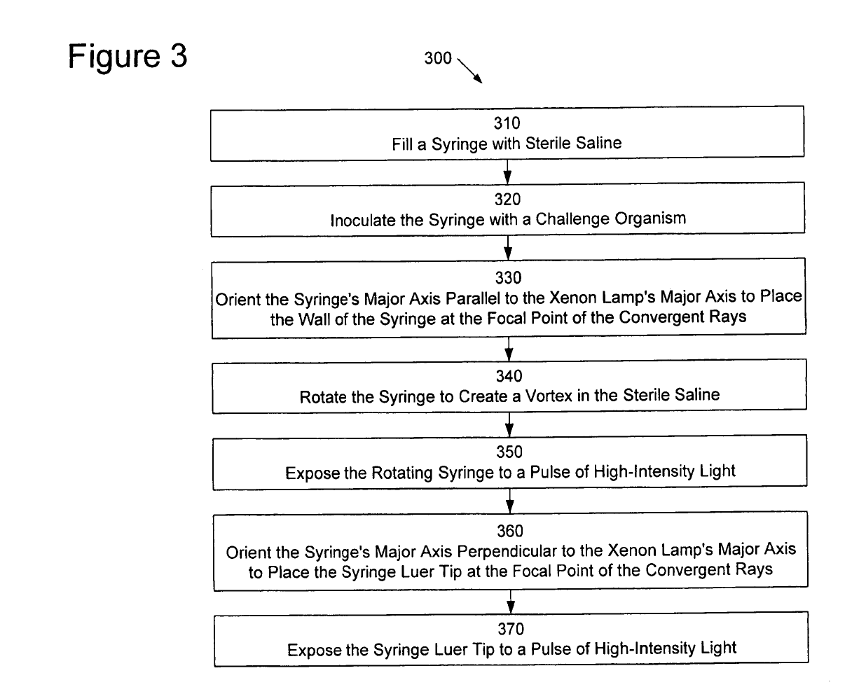

FIG. 3 is a flow chart that describes a terminal sterilization process used in the prototype system 200 shown in FIG. 2A and FIG. 2B. The process 300 begins at step 310 by filling a 12 cc syringe 263 with 10 cc of sterile saline (0.9% Sodium Chloride in water for injection). At step 320, the tester inoculates the sterile saline solution in the syringe 263 with a challenge organism, Bacillus pumilis at a level of 1×10⁶ to 1×10¹⁰ spores per device. The tester performs both the filling (step 310) and the inoculating (step 320) using aseptic technique.

The rotation and vortex generation unit 260 and syringe holder 262 retain and orient the syringe 263, at step 330, to align the major axis of the syringe 263 parallel to the major axis of the pulsed light source (xenon) lamp 252. This orientation places the sidewall of the syringe 263 at or near the focal point of the convergent rays of the pulsed light source (xenon) lamp 252. At step 340, the rotation and vortex generation unit 260 is the mechanical control that rotates the syringe 263 at a rate to induce the creation of a vortex within the inoculated saline solution held in the syringe 263.

The centrifugal force created by the vortex causes the spores to migrate toward the sidewall of the syringe 263. Furthermore, since the fluid does not entirely fill the syringe 263, the vortex also displaces the fluid contained in the small diameter luer tip, or opening, of the syringe 263 so that any spores contained in the fluid will likely migrate toward the sidewall of the syringe 263. Displacing the spores to the sidewall of the syringe 263 is advantageous because the spores are closer to the light source thus increasing the effectiveness, at step 350, of exposing the syringe 263 to the pulsed high-intensity light. The effectiveness of the exposure increases because light energy decreases as the light moves through the syringe 263 due to rays diverging as they move away from the focal point and diffracting as they pass through the fluid contained in the syringe 263. In one exemplary embodiment, 1100 revolutions per minute is a rate that induces the creation of a vortex. At 1100 revolutions per minute, the syringe 263 rotates around its' major axis 2.67 times during each 200 millisecond pulse of the pulsed light source (xenon) lamp 252. Thus, for each pulse of the pulsed light source (xenon) lamp 252, the sidewall of the syringe 263 receives multiple direct exposures to the high-intensity light.

At step 360, a magazine holder 265 orients the magazine 264 and syringes, such as syringe 263, to align the major axis of the syringes perpendicular to the major axis of the pulsed light source (xenon) lamp 252. This orientation places the luer tip, or opening, of the syringes at the focal point of the convergent rays of the pulsed light source (xenon) lamp 252. In one exemplary embodiment, the magazine 264 accommodates up to four syringes. In this orientation, the syringes do not rotate. At step 370, exposing the luer tip, or opening, of syringes to the pulsed high-intensity light, when combined with the rotating exposure (step 350), assures full exposure of the luer tip, or opening, of the syringes to the light source.

A test sample of syringes subjected to the process 300 and evaluated by an independent microbiological laboratory show a reduction of colony forming units of Bacillus pumilis between 1 and 10 log. Testing indicates that the level of reduction is dependent upon the total energy delivered during exposure, that is, the number of pulses applies to the test sample. Thus, the prototype system illustrated in FIG. 2A and FIG. 2B and described in FIG. 3 demonstrates that the process 300 is viable for use in the terminal sterilization of syringes containing saline inoculated with very high levels of a challenge organism. The selection of Bacillus pumilis was advantageous because it is widely used as a challenge organism in sterilization process development and validation and because an extensive body of literature using Bacillus pumilis exists in the area of sterilization by gamma irradiation.

In another exemplary embodiment of the process 300, the prototype terminal sterilization system 200 combines the orienting steps (step 330 and step 360) and combines the exposing steps (step 350 and step 370). In this exemplary embodiment, the combined orienting step places the container 141 in a chamber, such as the flashlamp control 140, shown in FIG. 1, to place the sidewall of the container 141 at the focal point of the convergent rays of the pulsed light source (xenon) lamp 142 and the tip of the container 141 at the focal point of the convergent rays of the lamp 144. The mechanical control 130 rotates the container 141 at a rate to induce the creation of a vortex within the fluid held in the container 141. In one exemplary embodiment, the combined exposing step staggers the flash from the individual lamps. In another exemplary embodiment, the combined exposing step will simultaneously expose the container 141 to multiple pulses of high-intensity light to assure full exposure of the sidewall and tip of the container 141 to the light source.

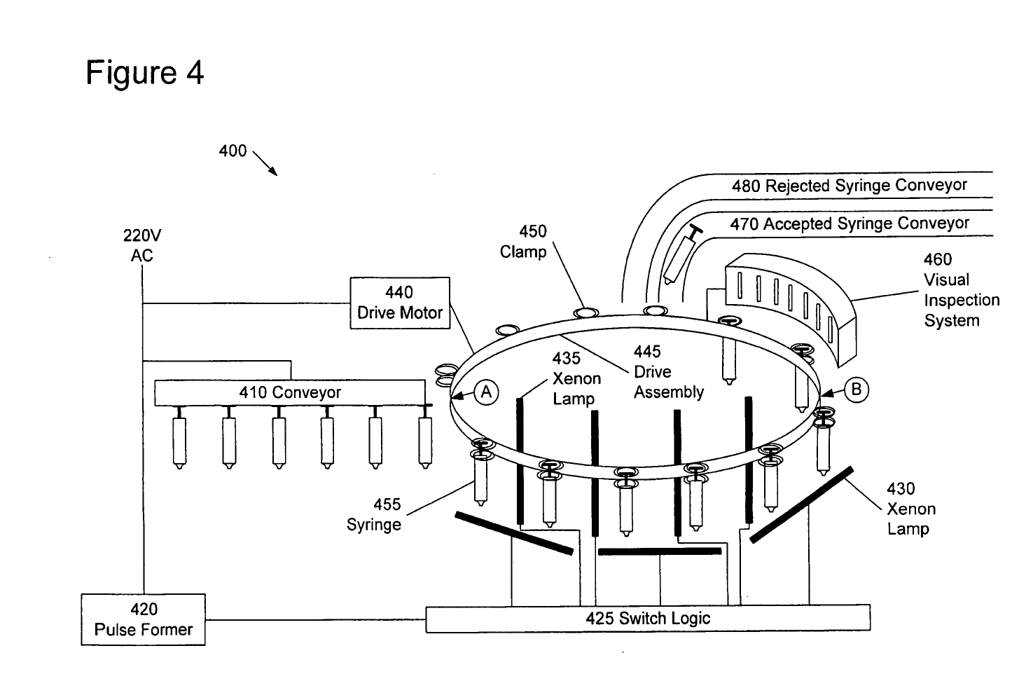

The prototype system 200, as shown in FIG. 2A and FIG. 2B, is not suitable for continuous production of terminally sterilized syringes or other containers or devices. At best, the prototype system 200 may terminally sterilize approximately 1-2 syringes per minute and uses a process that is very labor intensive. In contrast, a production line system must terminally sterilize syringes at varying line speeds. FIG. 4 illustrates the components for a production line exemplary embodiment of the terminal sterilization system 100 shown in FIG. 1. The production system 400 is suitable for terminally sterilizing syringes at varying line speeds. In other exemplary embodiments, the line speed may vary depending on the capability and features built into the machine, and the requirements dictated by the process or the material being sterilized.

As shown in FIG. 4, the production system 400 receives syringes from a conveyor 410, processes the syringes on a drive assembly 445, and releases the syringes to either an accepted syringe conveyor 470 or rejected syringe conveyor 480. An electrical source, such as a 120-volt or 220-volt alternating current, provides the power necessary to run the conveyor 410, drive motor 440, accepted syringe conveyor 470, and rejected syringe conveyor 480.

A clamp 450 is the means for transferring each syringe 455 from the conveyor 410 to the drive assembly 445 and from the drive assembly 445 to either the accepted syringe conveyor 470 or the rejected syringe conveyor 480. The clamp 450 also holds and retains each syringe 455 throughout the terminal sterilization process and, in concert with the drive assembly 445, is the means for rotating the syringe 455 to induce the creation of a vortex within the fluid held in the syringe 455.

The drive motor 440 is the mechanical control that rotates the drive assembly 445. The drive assembly 445 engages the means for rotating the syringe 455, such as a drive belt, gear, chain, or similar mechanism, at a start point A after grasping the syringe 455. The drive assembly 445 disengages the means for rotating the syringe 455 at a stop point B before approaching the visual inspection system 460. In one exemplary embodiment, the rotation of the drive assembly 445 is in a counter-clockwise direction. In another exemplary embodiment, the rotation of the drive assembly 445 is in a clockwise direction.

The electrical source also provides the power necessary to run a pulse former 420 and switch logic 425. The pulse former 420 functions similar to the pulse former 122 shown in FIG. 1 and pulse forming network module 230 shown in FIG. 2A and FIG. 2B. The pulse former 420 periodically generates an electrical pulse for a given duration of time. In one exemplary embodiment, the duration of the pulse is 200 milliseconds and the period is three pulses per second. The switch logic 425 controls the routing of the pulse to each vertical pulsed light source (xenon) lamp 435 and each horizontal pulsed light source (xenon) lamp 430. The vertical pulsed light source (xenon) lamp 435 and horizontal pulsed light source (xenon) lamp 430 differ from lamp housing 251 shown in FIG. 2A and FIG. 2B for the sake of occupying less acreage. The vertical pulsed light source (xenon) lamp 435 and horizontal pulsed light source (xenon) lamp 430 comprise two smaller co-axial lamps, with reflectors that provide a single convergent focal point for the rays from both lamps. For simplicity, FIG. 4 does not show the individual components such as a reflector and quartz window. FIG. 4 also does not show a reflector similar to the supplemental reflector 255 shown in FIG. 2A and FIG. 2B. However, the interior of the enclosure for the production system will include a surface that performs a function similar to the supplemental reflector 255. In one exemplary embodiment, the fabrication of the surface will include spectral aluminum sheet metal.

The visual inspection system 460 is a means for determining whether the fluid in the syringe 455 contains an acceptable level of particulate matter. The visual inspection system 460 includes automated inspection systems, manual inspection systems, human inspection systems, and photographic inspection systems that detect the presence of particulate matter with various sizes, including microscopic organisms, and objects visible to the human eye. If the level is acceptable, the visual inspection system 460 commands the drive assembly 445 to release the clamp 450 and drop the syringe 455 onto the accepted syringe conveyor 470. If the level is not acceptable, the visual inspection system 460 commands the drive assembly 445 to release the clamp 450 and drop the syringe 455 onto the rejected syringe conveyor 480.

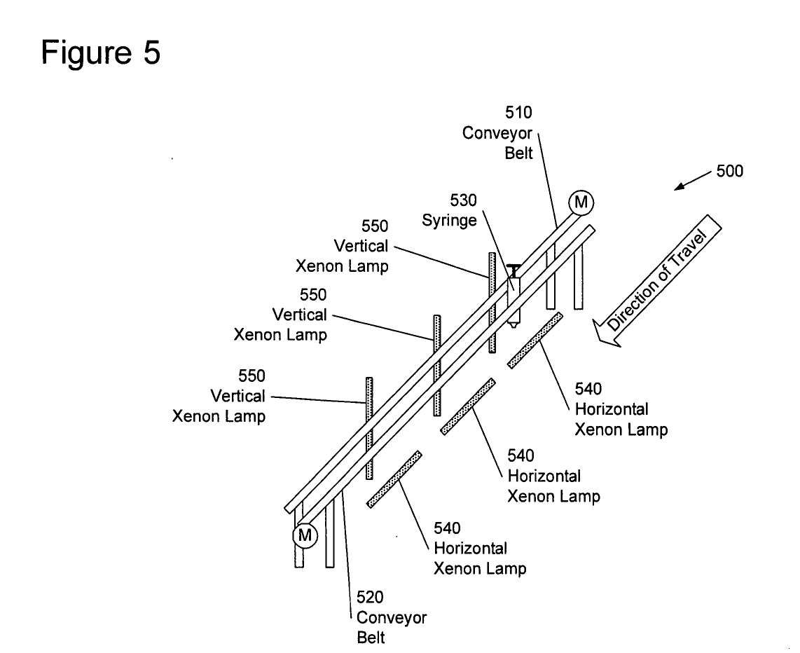

FIG. 5 shows a portion of a production system 500 similar to the production system 400 shown in FIG. 4. The portion of the production system 500 shown in FIG. 5 is a linearly-oriented exemplary embodiment of the portion of the production system 400 shown in FIG. 4 from the start point A to the stop point B. The remaining portions of the production system 500 shown in FIG. 5 are not shown or described, but are similar to those shown in FIG. 4.

As shown in FIG. 5, the production system 500 receives a syringe 530 at the beginning of the direction of travel on a set of conveyor belts 510, 520. The syringe 530 is held in place by a first conveyor belt 510 and a second conveyor belt 520. Each conveyor belt is independently controlled by a motor M. In one exemplary embodiment, the motors M are configured to rotate the first conveyor belt 510 in the opposite direction of the second conveyor belt 520. A variance in the speed differential between the rate of rotation of the first conveyor belt 510 and the rate of rotation of the second conveyor belt 520 causes the syringe 530 to rotate along its central axis while moving down the conveyor belts 510, 520 in the direction of travel. The variance of the speed differential not only controls the rate of rotation of the syringe 530 along its central axis, but also the dwell time for the syringe 530 in front of the horizontal xenon lamps 540 and the vertical xenon lamps 550. Thus, the set of conveyor belts 510, 520 are a means for transferring the syringe 530 and a means for rotating the syringe 530 to create a vortex in the fluid held by the syringe 530. The horizontal xenon lamps 540 and vertical xenon lamps 550 function similar to the horizontal pulsed light source (xenon) lamps 430 and vertical pulsed light source (xenon) lamps 435 as shown in FIG. 4.

Although the disclosed exemplary embodiments describe a fully functioning method and apparatus for terminal sterilization using pulsed high-intensity light, the reader should understand that other equivalent exemplary embodiments exist. Since numerous modifications and variations will occur to those reviewing this disclosure, the method and apparatus for terminal sterilization using pulsed high-intensity light is not limited to the exact construction and operation illustrated and disclosed. Accordingly, this disclosure intends all suitable modifications and equivalents to fall within the scope of the claims.

We claim:

1. A method for terminal sterilization, comprising:
orienting a container in relation to at least one flashlamp, the container including a wall having an outer surface and an inner surface, and holding a fluid;
creating a vortex in the fluid;
generating from said at least one flashlamp at least one pulse of high-intensity light in a broad spectrum; and
exposing the container to said at least one pulse of high-intensity light.

2. The method of claim 1, the orienting of the container further comprising:
aligning a major axis of the container parallel to a major axis of a first flashlamp of said at least one flashlamp; and
placing a first focal point of convergent rays of the first flashlamp at or near the inner surface of the wall of the container.

3. The method of claim 2, wherein the first focal point is a portion of the fluid that touches the inner surface of the wall of the container.

4. The method of claim 2, wherein the first focal point is a center of mass of the container.

5. The method of claim 2, wherein the first focal point is a center of the vortex.

6. The method of claim 1, the orienting of the container further comprising:
aligning a major axis of the container perpendicular to a major axis of a second flashlamp of said at least one flashlamp; and
placing a second focal point of convergent rays of the second flashlamp at or near the inner surface of the wall of the container at a tip, the tip located at an end of the container.

7. The method of claim 6, wherein the second focal point is a portion of the fluid that touches the inner surface of the container at the tip.

8. The method of claim 6, wherein the second focal point is a center of mass of the container.

9. The method of claim 6, wherein the second focal point is a center of the vortex.

10. The method of claim 1, the orienting of the container further comprising:
positioning the container between said at least one flashlamp and a supplemental reflector,
wherein the supplemental reflector reflects divergent rays toward the container, thereby increasing the energy input to the container.

11. The method of claim 1, the creating of the vortex further comprising:
rotating the container,
wherein a rate of the rotation creates the vortex.

12. The method of claim 11, wherein the rate is approximately 1100 revolutions per minute.

13. The method of claim 11, wherein the rate obtains at least one revolution of the container during a duration of said at least one pulse of high-intensity light.

14. The method of claim 1, wherein a material composition of the container has a transmission coefficient that allows an effective amount of said at least one pulse of high-intensity light to penetrate the container.

15. The method of claim 1, wherein a material composition of the container has a transmission coefficient that allows penetration of said at least one pulse of high-intensity light sufficient to provide a level of energy that is lethal to a viable organism inside the container.

16. The method of claim 1, wherein the fluid is a liquid.

17. The method of claim 1, wherein the fluid is a pharmaceutical drug or a non-pharmaceutical product.

18. The method of claim 1, wherein the container includes a syringe, vial, test tube, bottle, boxes, bags, or the like capable of holding the fluid.

19. The method of claim 1, wherein a wavelength of said at least one pulse of high-intensity light is in a spectral region of interest of approximately 254 nanometers.

20. The method of claim 1, wherein a wavelength of said at least one pulse of high-intensity light is in a spectral region of interest in the range of approximately 150 nanometers to approximately 2600 nanometers.

21. The method of claim 1, wherein a duration of said at least one pulse of high-intensity light is variable.

22. The method of claim 1, wherein said at least one pulse of high-intensity light is ultraviolet light or polychromatic light.

23. The method of claim 1, further comprising:
inspecting the fluid to detect the presence of particulate matter.

24. The method of claim 23, wherein the inspecting of the fluid occurs after the exposing of the container to said at least one pulse of high-intensity light.

25. The method of claim 23, wherein the inspecting of the fluid occurs after the creating of the vortex and before the vortex disappears.

26. The method of claim 1, wherein the container further includes an inner container comprising a wall having an outer surface and an inner surface, and wherein the fluid is held in a space between the inner surface of the wall of the container and the outer surface of the wall of the inner container.

27. The method of claim 26, wherein the vortex is a Taylor vortex.

28. The method of claim 26, the creating of the vortex further comprising:
rotating the container,
wherein a rate of the rotation of the container creates the vortex.

29. The method of claim 26, the creating of the vortex further comprising:
rotating the inner container in a direction opposite the rotation of the container,
wherein combination of the rate of the rotation of the container and a rate of rotation of the inner container creates the vortex.

30. The method of claim 26, the creating of the vortex further comprising:
rotating the inner container,
wherein a rate of the rotation of the inner container creates the vortex.

31. An apparatus for terminal sterilization, comprising:
at least one flashlamp;
a pulse formation means for generating at least one pulse of high-intensity light in a broad spectrum from said at least one flashlamp; and
a mechanical control means for orienting a container in relation to said at least one flashlamp, and rotating the container to create a vortex in a fluid held inside the container,
wherein exposure of the container and the fluid to said at least one pulse of high-intensity light terminally sterilizes the container and the fluid.

32. The apparatus of claim 31, wherein the mechanical control means further comprises:
an orienting means for aligning a major axis of the container parallel to a major axis of a first flashlamp of said at least one flashlamp, and placing a first focal point of convergent rays of the first flashlamp at or near an inner surface of a wall of the container.

33. The apparatus of claim 31, wherein the mechanical control means further comprises:
an orienting means for aligning a major axis of the container perpendicular to a major axis of a second flashlamp of said at least one flashlamp, and placing a second focal point of convergent rays of the second flashlamp at or near an inner surface of a wall of the container at a tip, the tip located at an end of the container.

34. The apparatus of claim 31, wherein the mechanical control means further comprises:
an orienting means for positioning the container between said at least one flashlamp and a supplemental reflector,
wherein the supplemental reflector reflects divergent rays toward the container, thereby increasing the energy input to the container.

35. The apparatus of claim 31, further comprising:
an inspection means for detecting the presence of particulate matter in the fluid to determine whether particulate matter is present in the fluid.
